(19) United States
(12) Patent Application Publication
(10) Pub. No.: US 2009/0240111 A1
(43) Pub. Date: Sep. 24, 2009

(54) Light Source and Lens Cleaner for Laparoscopic Surgery

(76) Inventors: Joshua Kessler, Danbury, CT (US); Jason Kunze, Warrenville, IL (US); Elliott Rouse, Evanston, IL (US); Garrett Green, Irvine, CA (US); Hariharan Subramanian, Chicago, IL (US)

Correspondence Address: Josh Kessler, 17 Marc Rd, Danbury, CT, 06810, US

(21) Appl. No.: 12/406,152

(22) Filed: Mar. 18, 2009

Related U.S. Application Data
(60) Provisional application No. 61/037,367, filed on Mar. 18, 2008.

(51) Int. Cl. A61B 1/012; A61B 1/06

(52) U.S. Cl. 600/155; 600/179

(57) ABSTRACT

An intraperitoneal cleaner to allow cleaning of a scope lens used in minimally invasive surgery is presented. Using existing super elastic alloys in combination with a standard spring, a purely mechanical cleaning action is provided, eliminating the reservoirs, vacuum pumps, or extra ports found in other cleaner designs. The scope body is inserted into the device prior to commencement of surgery, and the wiper of the cleaner rests just outside of the field of view of the lens of the scope. When debris or fluid obscures the field of view, a spring in the handle is compressed, allowing the cleaner to flex and provide a cleaning action.

A cordless laparoscopic light source using LEDs and a pair of aspherical condensing lenses to focus the resulting light to a usable point is presented. By using battery power, the entire device is considerably smaller than the current xenon light source standard. Additionally, fiber optic cords which restrict movement have been eliminated, and the inherent fire risk from a xenon source has been removed.

### CROSS-REFERENCE TO RELATED APPLICATIONS

Not Applicable.

### STATEMENT REGARDING FEDERALLY SPONSORED RESEARCH OF DEVELOPMENT

Not Applicable

### REFERENCE TO SEQUENCE LISTING, A TABLE, OR A COMPUTER PROGRAM LISTING COMPACT DISC APPENDIX

Not Applicable

### BACKGROUND OF THE INVENTION

In the field of minimally invasive surgery, visibility is a paramount requirement. However, due to the small size of the objective lenses (less than one centimeter) any droplet of liquid or moisture, or piece of tissue results in a dramatic compromise of the field of view. Currently the accepted solution is to pause the procedure, withdraw the scope, and clean the lens by hand prior to reinsertion.

Clearly this time consuming process is out of place in a cutting edge operating room, and an intraperitoneal cleaner has long been sought after. Numerous prior attempts employed fluid sprays, vacuum suctions, or cleaners introduced into the surgical area via a secondary port in the patient's abdomen, none of these devices found meaningful market acceptance.

Our cleaning invention is entirely novel, however, the following patents were discovered in our prior art search:

U.S. Pat. No. 6,755,782: Endoscope dirt remover
U.S. Pat. No. 6,447,446: Method and apparatus for cleaning an endoscope lens
U.S. Pat. No. 4,919,113: Method of cleaning scope end of endoscope and endoscope with scope end cleaning mechanism
U.S. Pat. No. 6,354,992: Automated laparoscopic lens cleaner
U.S. Pat. No. 5,400,767: Laparoscopic telescope lens cleaner and protector
U.S. Pat. No. 5,313,934: Lens cleaning means for invasive viewing medical instruments
U.S. Pat. No. 6,699,331: Endoscope cleaning device
U.S. Pat. No. 3,145,249: Endoscope window
U.S. Pat. No. 6,755,782: Endoscope dirt remover
U.S. Pat. No. 5,654,824: Portable self-cleaning mirror apparatus and method
U.S. Pat. No. 6,447,466: Method and apparatus for cleaning an endoscope lens
U.S. Pat. No. 4,919,113: Method of cleaning scope end of endoscope and endoscope with scope end cleaning mechanism
U.S. Pat. No. 6,354,992: Automated laparoscopic lens cleaner
U.S. Pat. No. 5,492,766: System and method for cleaning viewing scope lenses
U.S. Pat. No. 5,514,082: Retractable wipe for cleaning endoscopic surgical devices
U.S. Pat. No. 5,647,840: Endoscope having a distally heated distal lens
U.S. Pat. No. 6,712,757: Endoscope sleeve and irrigation device
U.S. Pat. No. 5,605,532: Fog free endoscope
U.S. Pat. No. 5,464,008: Laparoscope defogging
U.S. Pat. No. 5,382,297: Endoscope cleaning and defogging apparatus
U.S. Pat. No. 6,409,657: Cleaning device for cleaning view window of endoscope

Additionally, a fundamental need of visibility is a source of sufficient light. Currently xenon light sources are common and sufficient, however they are extremely expensive, large, loud, and in certain situations, present a fire hazard in the operating room. Additionally, the fiber optic delivery cable can severely limit mobility during a procedure. Our light device uses an array of high intensity LEDs and a novel short focal length focusing system to provide a high strength light, but in a device package small enough to attach directly to the scope, removing the cable, and the entire oven sized cart of supporting electronics that accompanies a xenon source. For the light, the following invention does specify using LEDs: U.S. Pat. No. 7,041,054, and we felt the following patents were similar, but that ours had a novel difference to each:

U.S. Pat. No. 5,888,194: Endoscope including an improved lighting apparatus
U.S. Pat. No. 6,260,994: Battery-powered light source arrangement for endoscope
U.S. Pat. No. 7,198,397: LED endoscope illuminator and methods of mounting within an endoscope
U.S. Pat. No. 6,814,699: Light source for borescopes and endoscopes

### BRIEF SUMMARY OF THE INVENTION

Our cleaning invention is a disposable intraperitoneal cleaner for scopes used in minimally invasive surgery. It slides on over the body of the scope, orienting a dual position wiper in the plane of, and in contact with, the lens, and an actuator on the proximal end, exterior to the patient. By moving a slide or depressing a button on the actuator, the wiper is allowed to move between positions, and in the process, removes fluid and debris from the lens. The wiper is constructed of super elastic material, so that it can deform between orientations numerous times without breakage, and the deformation imparts sufficient force to be an effective cleaning element.

Unlike the majority of the patents cited above, our device attaches to the scope itself, removing the need for additional ports, and operates mechanically, removing the need for sprays and suctions (and therefore reservoirs and pumps).

Our light invention is a battery operated array of high intensity LEDs focused with a novel dual short focal length lens system to consolidate the light and deliver it to a short segment of fiber optic that will attach to the existing light terminals on scopes in the market today. The weight of the batteries will be distributed in such a way that the center of gravity is on the scope body, thus providing ease of motion and balance during extended procedures.

### BRIEF DESCRIPTION OF THE SEVERAL VIEWS OF THE DRAWING

FIG. 1: Obtuse view of entire cleaning device showing wiper, body, and actuator

FIG. 2: Side view of wiper end of cleaner

FIG. 3: Angled view of light showing distal lens, exhaust fan vents, and battery orientation

FIG. 4: Cutaway side view of light showing lens orientation

### DETAILED DESCRIPTION OF THE INVENTION

The cleaner invention is easily reduced to three primary elements: the wiper (FIG. 1, “wiper”), body (FIG. 1, “body”), and actuator (FIG. 1, “actuator”).

The body is a tube (the majority of which can be only an arc rather than the full circumference), the inner diameter of which matches the exterior diameter of the scope intended to accept this device. The distal end of this tube is at an angle matching that of the distal end of the scope (typically between 90 and 135 degrees relative to the long axis of the body).

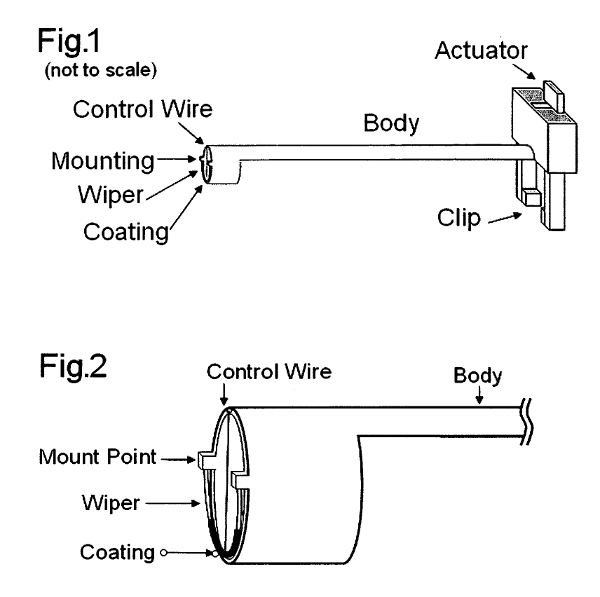

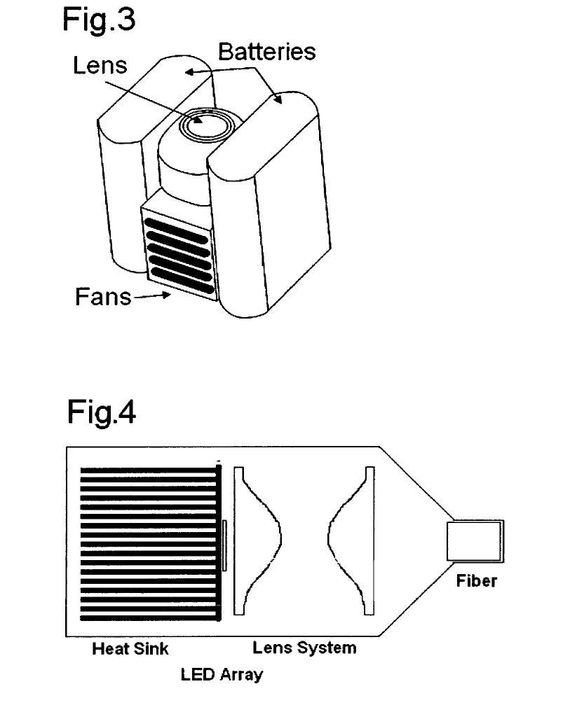

Upon the tube edge on the plane of the lens opening are two raised points (FIG. 2, “mount points”) into which is inserted and affixed with pressure, adhesive, or both, an arc of nitinol or other super elastic alloy. This arc lies in the plane of the lens opening, and in a resting position, occupies an arc of the circumference opposite the control wire channel mentioned later. This wiper is coated partially, or in full, with a softer material than the alloy to effect the cleaning (FIG. 2, “wiper coating”). This material can be hydrophobic, hydrophilic, or a combination of the two, depending on the particular operating environment, and the likely debris encountered therein.

Attached to the center of this wiper is a much finer wire (FIG. 1, “control wire”), also of a super elastic alloy, this wire then enters a channel within the wall of the primary body tube, which runs the entire length of the body, and is affixed to the actuator. At the lower portion of the actuator are two extensions that apply pressure to opposite sides of the scope body to hold the cleaner to the scope and prevent the two from moving independent of each other. For certain scope bodies, this can be supplemented with a plastic thumb “set” screw.

Within the actuator is a spring of sufficient force to deform the wiper so that it occupies the arc closest to the control wire channel, thus removing the control wire from the field of view. The spring should also be of a strength such that it can be compressed easily by an adult thumb. This compression then introduces slack to the control wire. Due to the inherent nature of the alloy in the wiper, it will then flex back to the opposite orientation, and effect a cleaning action during this process. When the spring is released, it will then reapply sufficient tension to withdraw the wiper.

The light invention consists of two ENFIS 5×5 LED arrays which together provide 700 Lumens. As LED technology is improving rapidly, this is by no means a rigid requirement, and the highest lumen/area device should be used, considering only those arrays with a white/close to white wavelength signature. To dissipate heat we use a 25×25×25 mm aluminum heat sink with two fans to provide forced convection. The most novel aspect of this device is the use of a pairing of aspherical condensing lenses to focus the LED light to a single point. Battery specifications cannot be outlined here as requirements are dependent on the LED arrays employed, and physical design is then dependent on the batteries.

What is claimed is:

1. A scope lens cleaner comprising:
A single tubular shaped device with a lens opening at the distal end and an actuator at the proximal end, into which is inserted a scope;
A wire sweeper having a semi-circular shape fastened to the distal end such that each end of the wire forms a fulcrum, said sweeper being constructed of super elastic shape memory alloy, and while alternating between deformed and original shapes, slides across the plane of, and remains in contact with, the scope lens;
A wire connector running from said wire sweeper to the proximal end, and;
A spring mechanism within the proximal end of the device that provides a mechanical control of the positioning of said wire connector, positioned more proximally than the trocar permitting access to the patient.

2. A scope lens cleaner according to claim 1, wherein said distal end with a lens opening being formed in shapes conforming to the varying angled heads and diameter sizes of different scopes.

3. A scope lens cleaner according to claim 1, said wire sweeper having a semi-circular shape fastened to the said cap such that each end forms a fulcrum, said wire sweeper being coated by or attached to a thin wiper material used to more effectively sweep dirt from said scope lens.

4. A scope lens cleaner according to claim 3, wherein said wiper material used to more effectively sweep dirt from the scope lens, said wiper material being made of flexible material that is either hydrophobic, hydrophilic, or a combination of the two.

5. A scope lens cleaner according to claim 3, wherein said wiper material used to more effectively sweep dirt from said scope lens, said wiper material is bonded to said thin wiper material in a way such that both retain elasticity.

6. A scope lens cleaner according to claim 3, wherein said wiper material used to more effectively sweep dirt from said scope lens, said wiper material is bonded to said thin wiper material in a manner such that both remain flexible.

7. A scope lens cleaner according to claim 3, wherein said wire connector from said wire sweeper to the distal end of the scope, said wire connector to be attached to said wire sweeper directly or by an optically transparent wire.

8. A scope lens cleaner according to claim 3, wherein said control mechanism being attached in a way such that said mechanical control is easily operated during normal usage of said scope.

9. A scope lens cleaner according to claim 3, wherein said mechanical control controls the positioning of said wire connector, said mechanical control allows user to set the length of said wire connector between said distal and proximal ends of the scope.

10. A scope lens cleaner according to claim 3, wherein the scope is a laparoscope or endoscope.

11. A cordless laparoscopic light source comprising:
One or more LED arrays;
Heat sink;
One or more fans to provide forced convection;
A pair of aspherical condensing lenses to focus the light, and;
Sufficient battery power to run the LEDs for a designated time.

12. A cordless laparoscopic light source according to claim 11, wherein said battery power is not proximal to the body of the device, but rather worn by the user and delivered via wire.

13. A cordless laparoscopic light source according to claim 11, wherein said power is supplied by batteries lasting less than the designated time, but is supplied in a number of batteries to be swapped out, so that the combined result reaches the designated time.

14. A cordless laparoscopic light source according to claim 11, wherein the convection is supplied by a heat pipe or chimney.

15. A cordless laparoscopic light source according to claim 11, wherein the LED arrays are heat efficient to the degree wherein the heat sink and fan are not necessary.
